(19) United States
(12) Patent Application Publication
(10) Pub. No.: US 2025/0044242 A1
(43) Pub. Date: Feb. 6, 2025

(54) SYSTEMS AND METHODS FOR NON-INVASIVE GLUCOSE MONITORING

(71) Applicant: KING FAHD UNIVERSITY OF PETROLEUM AND MINERALS, Dhahran (SA)

(72) Inventors: Yaqub Alhussain MAHNASHI, Dhahran (SA); Khurram Karim QURESHI, Dhahran (SA); Abdullah Ali AL-SHEHRI, Dhahran (SA); Hussein Mahmoud ATTIA, Dhahran (SA)

(73) Assignee: KING FAHD UNIVERSITY OF PETROLEUM AND MINERALS, Dhahran (SA)

(21) Appl. No.: 18/364,940

(22) Filed: Aug. 3, 2023

(51) Int. Cl. G01N 22/00; A61B 5/145; G01N 33/49

(52) U.S. Cl. CPC G01N 22/00; A61B 5/14532; G01N 33/49; A61B 5/0507

(57) ABSTRACT

A system for monitoring glucose concentration that includes a sample holder configured to contain a sample and a radio frequency transmitter configured to emit microwave radiation through the sample. The radio frequency transmitter comprising a radio frequency generator and a first antenna. The radio frequency receiver is configured to receive microwave radiation transmitted from the radio frequency transmitter and passed through the sample, and convert the microwave radiation passed through the sample to an analog signal. The radio frequency receiver comprising a second antenna. The system also includes an analog readout circuit configured to receive the analog signal and process the analog signal into a modified analog signal, and a digital signal processing circuit configured to receive the modified analog signal, convert the modified analog signal into a digital signal, and determine the glucose concentration of the sample from the digital signal.

### STATEMENT OF ACKNOWLEDGMENT

The support received from King Fahd University of Petroleum & Minerals (KFUPM) and the Center of Communication Systems and Sensing under project #INCS2106 is gratefully acknowledged.

### STATEMENT OF PRIOR DISCLOSURE BY AN INVENTOR

Aspects of the present disclosure are described in Y. Mahnashi, et al., “Design and Experimental Validation of a Noninvasive Glucose Monitoring System Using RF Antenna-Based Biosensor,” in IEEE Sensors Journal, vol. 23, no. 3, pp. 2856-2864.

### BACKGROUND

### Technical Field

The present disclosure is directed to systems and methods for non-invasive glucose monitoring

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

The widespread presence of diabetes has been highly associated with an unhealthy diet and limited physical activity. According to the World Health Organization (WHO), there was a global increase in diabetes among adults from 4.7% in the year 1980 to 8.5% in the year 2014. Therefore, a diabetes medical management plan is essential help patients avoid the complications of diabetes, including cardiovascular disease, kidney damage, blindness, increased risk of stroke, and foot damage. Currently, most blood glucose monitoring systems rely on ambulatory devices that involve sampling blood from a finger using a lancet needle. However, in addition to being painful and inducing anxiety in patients, these ambulatory devices exhibit high error rates in the range of about 15% to about 20%. Moreover, ambulatory devices perform capillary glucose measurement that is known to be inaccurate. Also, existing ambulatory devices involve invasiveness, which is the main drawback that needs to be mitigated or eliminated to avoid harmful effects on patients. Furthermore, it is well known that commercially available devices for continuous blood glucose monitoring are costly and only last for about two weeks. Therefore, a more reliable, low-cost, long-lasting, and efficient technology is needed to ensure continuous blood glucose monitoring without harming patients psychologically or physically.

Recently, several non-invasive glucose monitoring techniques have been considered. Examples include exhalation breath and biological body fluids analysis in addition to many spectroscopic techniques that are unsuitable for continuous monitoring. However, these techniques exhibit notable drawbacks that may affect the accuracy of the measured results. For example, the techniques utilizing exhalation breath samples are inefficient and inaccurate. Accordingly, there is a need for a portable and efficient non-invasive glucose monitoring system and method.

### SUMMARY

In an exemplary embodiment, a system for monitoring glucose concentration is disclosed. The system includes a sample holder configured to contain a sample and a radio frequency transmitter configured to emit microwave radiation through the sample. The radio frequency transmitter comprising a radio frequency generator and a first antenna. The system may further include a radio frequency receiver configured to receive microwave radiation transmitted from the radio frequency transmitter and passed through the sample, and convert the microwave radiation passed through the sample to an analog signal. The radio frequency receiver includes a second antenna. The first and second antennas are microstrip antennas having a sample side and an outward side. The microstrip antennas include a ground plate, a dielectric substrate disposed on the ground plate, and a conductive pattern disposed on the dielectric substrate. The conductive pattern on the sample side of the microstrip antenna includes a rectangular patch with a plurality of slots disposed on the patch. The plurality of slots are configured to expose regions of the dielectric substrate. The rectangular patch is symmetrical about a long axis. The long axis defines first and second sides of the sample side of the rectangular patch. The sample side of the rectangular patch includes a plurality of slots including two horizontal slots symmetrically oriented at a top half of the rectangular patch, two horizontal slots symmetrically oriented at a bottom half of the rectangular patch, and a vertically oriented slot disposed on the long axis. The system further includes an analog readout circuit configured to receive the analog signal and process the analog signal into a modified analog signal, and a digital signal processing circuit configured to receive the modified analog signal, convert the modified analog signal into a digital signal, and determine the glucose concentration of the sample from the digital signal.

In another exemplary embodiment, a system for monitoring glucose concentration is disclosed. The system includes a radio frequency generator, a first antenna configured to transmit microwave radiation through a sample, and a second antenna configured to receive microwave radiation transmitted by the first antenna and convert the microwave radiation into an analog signal. The system further includes an analog readout circuit configured to receive the analog signal and process the analog signal into a modified analog signal, and a digital signal processing circuit configured to receive the modified analog signal, convert the modified analog signal into a digital signal, and determine the glucose concentration of the sample from the digital signal.

In yet another exemplary embodiment, a method for monitoring the glucose concentration is disclosed.

The method includes generating microwave radiation and transmitting the microwave radiation from a first antenna, through a sample. The method further includes receiving the microwave radiation from the first antenna with a second antenna, converting the microwave radiation received by the second antenna into an analog signal, processing the analog signal into a modified analog signal, and converting the modified analog signal into a digital signal. The method further includes determining the glucose concentration of the sample from the digital signal, transmitting the glucose concentration to a display device, and outputting the glucose concentration on the display device.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 illustrates a block diagram of a system for monitoring glucose concentration, according to certain embodiments.

FIG. 2 illustrates a detailed block diagram of the system for monitoring glucose concentration, according to certain embodiments.

FIG. 3A to FIG. 3D show a dual-band microstrip antenna, according to certain embodiments.

FIG. 4 shows a graphical representation of simulated scattering parameters of two antennas when placed 7 cm apart facing each other, and when there are no samples between the two antennas, according to certain embodiments.

FIG. 5 shows a three-dimensional radiation pattern (directivity) at 5.7 GHz of sensing antennas setup, according to certain embodiments.

FIG. 6 shows peak electric field (near-field) distribution at 5.7 GHz of the sensing antennas setup, according to certain embodiments.

FIG. 7 shows a graphical representation of an electric field calculated along a virtual dash-line between a first antenna and a second antenna, according to certain embodiments.

FIG. 8 shows a schematic diagram of an analog readout circuit, according to certain embodiments.

FIG. 9 describes an experimental setup of the system for monitoring glucose concentration, according to certain embodiments.

FIG. 10 shows a graphical representation of Direct Current (DC) output voltage trend versus glucose concentration, according to certain embodiments.

FIG. 11 shows a graphical representation illustrating a response of the DC output voltage to different power levels for glucose-free samples and 5 g/dL glucose concentration samples, according to certain embodiments.

FIG. 12 shows a graphical representation of a voltage range between two samples including glucose-free samples and 5 g/dL glucose concentration samples versus different input power levels, according to certain embodiments.

FIG. 13 illustrates a flowchart for monitoring glucose concentration, according to certain embodiments.

FIG. 14 is an illustration of a non-limiting example of details of computing hardware used in the computing system.

FIG. 15 is an exemplary schematic diagram of a data processing system used within the computing system.

FIG. 16 is an exemplary schematic diagram of a processor used with the computing system.

FIG. 17 is an illustration of a non-limiting example of distributed components which may share processing with the controller.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Aspects of this disclosure are directed to a portable non-invasive glucose monitoring system and method. The glucose monitoring system is an end-to-end low-cost microwave-based sensing system capable of detecting glucose level in aqueous solutions through a non-invasive scheme.

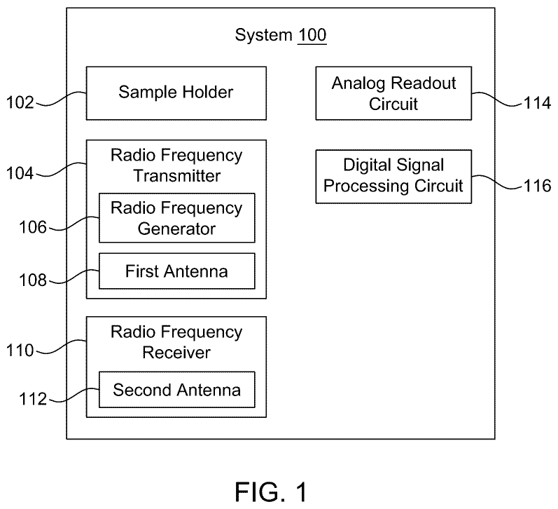

FIG. 1 illustrates a block diagram of a system 100 for monitoring glucose concentration, according to certain embodiments.

The system 100 includes a sample holder 102 and a radio frequency transmitter 104. The sample holder 102 is configured to contain a sample. In examples, the sample may be a glucose-water solution. The sample may also comprise a cross section of the human body which may be utilized as a sample without excision or otherwise removing it from the human body. The sample may also comprise human blood or a solution thereof. The radio frequency transmitter 104 may comprise a radio frequency generator 106 and a first antenna 108. The system 100 may include a radio frequency receiver 110. The radio frequency receiver 110 may include a second antenna 112. The first antenna 108 and the second antenna 112 are two radio frequency microstrip patch antennas which may function as biosensors. In examples, the radio frequency microstrip patch antennas resonate at 5.7 GHz and are fabricated using an FR-4 substrate. The radio frequency microstrip patch antennas may also resonate at 2.5 GH. The radio frequency microstrip patch antennas may also resonate at 5.7 GHZ and 2.5 GHz simultaneously. The radio frequency microstrip patch antennas may also resonate at any frequency in the range of 0.1 GHz to 11 GHz. Further, the first antenna 108 and the second antenna 112 are microstrip antennas having a sample side and an outward side. The microstrip antennas may be lightweight, small size, low cost, simple feeding structure, conformal nature, and can be shaped and placed on almost any surface.

The microstrip antennas may further comprise a ground plate, a dielectric substrate disposed on the ground plate, and a conductive pattern (also referred to as a radiating patch) disposed on the dielectric substrate. In examples, the conductive pattern may be printed on one side of the dielectric substrate. The conductive pattern may also be disposed on two sides of the dielectric substrate and/or on two sides of the radio frequency microstrip patch antennas. The ground plate may be on the opposite side of the dielectric substrate. The conductive pattern on the sample side of the microstrip antenna includes a rectangular patch with a plurality of slots disposed on the patch. The plurality of slots are configured to expose regions of the dielectric substrate.

The conductive pattern may be disposed on one or both sides of one or more microstrip antennas. The dielectric substrate may comprise any material which is an electrical insulator and may be polarized through the introduction of an electrical field. The microstrip antennas may comprise one or more dielectric substrates of the same or varying types which may function independently or in concert with one another.

The rectangular patch may be symmetrical about a long axis. The long axis defines first and second sides of the sample side of the rectangular patch. The sample side of the rectangular patch may include a plurality of slots including two horizontal slots symmetrically oriented at a top half of the rectangular patch, two horizontal slots symmetrically oriented at a bottom half of the rectangular patch, and a vertically oriented slot disposed on the long axis. The conductive pattern on the outward side of the microstrip antenna may include a rectangular patch with a single slot disposed on the patch. The slot is configured to expose a region of the dielectric substrate. The rectangular patch may be symmetrical about a long axis. The long axis defines first and second sides of the outward side of the rectangular patch. The slot may be positioned across the first side and the second side of the rectangular patch. The two horizontal slots symmetrically oriented at the top half of the rectangular patch are preferably the same size as one another. The two horizontal slots symmetrically oriented at the bottom half of the rectangular patch are preferably the same size as one another. The vertically oriented slot disposed on the long axis is preferably larger than any of the horizontal slots on the rectangular patch. The microstrip antennas may operate at multiple frequency bands (for example, dual, triple, etc.) by controlling the shape and size of the conductive pattern. The microstrip antennas are preferably configured to operate at the resonant frequencies of 2.5 GHz and 5.7 GHz. The system 100 further comprises an analog readout circuit 114 and a digital signal processing circuit 116.

The plurality of slots may be disposed on one or more sides of one or both microstrip antennas. The plurality of slots may be comprised of fewer or more individual slots on one or both sides of one or both microstrip antennas. The plurality of slots may be of any size, shape, and oriented with any relationship to one another such that the first antenna 108 may pass electromagnetic radiation of a desired wavelength to the second antenna 112.

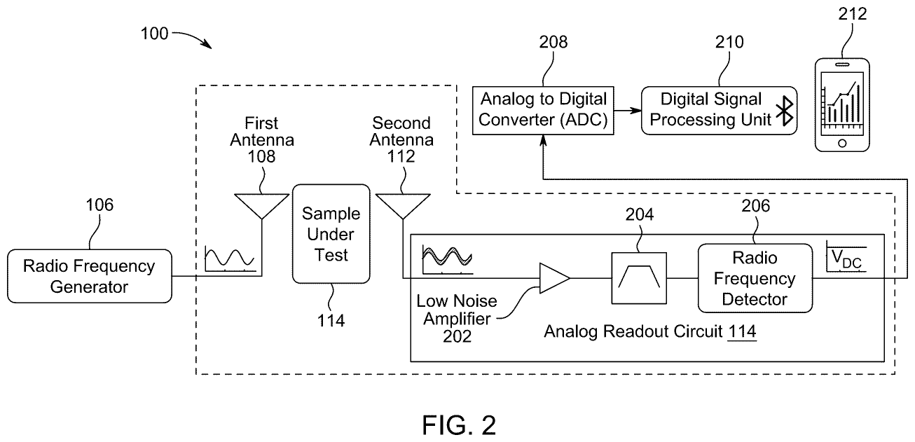

FIG. 2 illustrates a detailed block diagram of the system 100 for monitoring the glucose, according to certain embodiments.

In the example shown in FIG. 2, the analog readout circuit 114 includes a low noise amplifier 202, a band-pass filter 204, and a radio frequency detector 206. The low noise amplifier 202 is configured to operate between 0.4 GHz and 11 GHz. The band-pass filter 204 is configured to operate in the frequency range of 5.7 GHz to 6.0 GHz. The radio frequency detector 206 includes a high precision wideband radio frequency power detector. The high precision wideband radio frequency power detector is configured to detect radio frequencies in the range of 0.3 GHz to 7 GHz. Any other component of the analog readout circuit 114 may also be configured to resonate at any frequency which the radio frequency microstrip patch antennas may resonate at.

The digital signal processing circuit 116 includes an analog to digital signal converter 208 (also referred to as analog to digital converter (ADC)), a digital signal processing unit 210, a wireless communication unit, and a display device 212. The digital signal processing unit 210 may be a microcontroller. Further, the wireless communication unit comprises a Bluetooth transmitter. Furthermore, the display device 212 comprises a Bluetooth receiver and a screen.

According to an implementation, the radio frequency generator 106 may be configured to generate microwave radiation. In some implementations, the radio frequency transmitter 104 is configured to emit microwave radiation through a sample (i.e, sample under test). The radio frequency transmitter 104 may transmit the microwave radiation from the first antenna 108, through the sample.

According to another implementation, the radio frequency generator 106 may be configured to generate other forms of electromagnetic radiation such as very high frequency radio waves (“VHF”) or far infrared electromagnetic radiation (“FI”).

The radio frequency receiver 110 may be configured to receive the microwave radiation transmitted from the radio frequency transmitter 104 and passed through the sample. In examples, the radio frequency receiver 110 may receive the microwave radiation from through the second antenna 112. In an implementation, the radio frequency receiver 110 is configured to convert the microwave radiation passed through the sample and received by the second antenna 112 into an analog signal. The analog readout circuit 114 is configured to receive the analog signal and condition the analog signal. In an implementation, the analog readout circuit 114 is configured to process the analog signal into a modified analog signal. The digital signal processing circuit 116 may be configured to receive the modified analog signal, convert the modified analog signal into a digital signal, and determine the glucose concentration of the sample from the digital signal. The digital signal processing circuit 116 may also determine the glucose concentration of a sample. The digital signal processing circuit 116 is configured to wirelessly transmit the glucose concentration to the display device 212, for example, via the wireless communication unit (such as, Bluetooth transmitter). The display device 212 is configured to visually output the glucose concentration.

The analog signal may comprise any voltage, current, or measurable physical quantity that continuously and infinitely varies in accordance with a time-varying parameter. The analog signal may comprise one or more types of signal and may be converted between various forms within the digital signal processing circuit. The analog signal may be stagnant or fluctuating.

In some implementations, the first antenna 108 is configured to transmit microwave radiation through a sample. The second antenna 112 is configured to receive microwave radiation transmitted by the first antenna 108 and convert the microwave radiation into an analog signal. The analog readout circuit 114 is configured to receive the analog signal and process the analog signal into a modified analog signal. The digital signal processing circuit 116 is configured to receive the modified analog signal, convert the modified analog signal into a digital signal, and determine the glucose concentration from the digital signal.

The digital signal may comprise any signal that represents data as a sequence of discrete values which may be generated, stored, used by, and/or communicated by a digital device such as a personal computer. The digital signal may comprise one or more types of signal and may be converted between various forms. The digital signal may be stagnant or fluctuating.

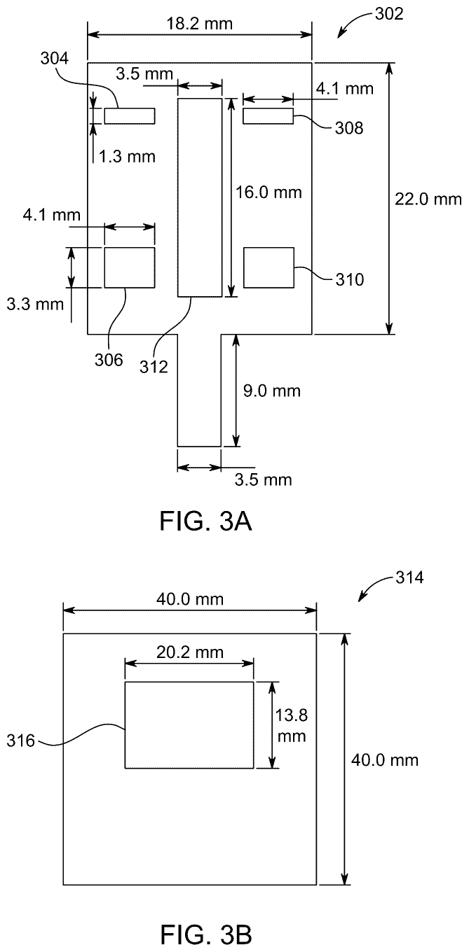

FIG. 3A to FIG. 3D show a dual-band microstrip antenna, according to certain embodiments. FIG. 3A shows a top view 302 of the dual-band microstrip antenna with five rectangular slots (represented by reference numerals 304, 306, 308, 310, and 312, respectively). FIG. 3B shows a back view 314 of the dual-band microstrip antenna. The back view 314 of the dual-band microstrip antenna shows a ground plate with a large rectangular slot (represented by reference numeral 316). FIG. 3C shows a perspective view 318 of the dual-band microstrip antenna. FIG. 3D shows a fabricated antenna proto-type 320 with a mock sample placed between two microstrip antenna antennas.

In an implementation, the dual-band microstrip antennas are adopted as a sensing element. The commercial electromagnetic software CST Microwave Studio may be employed to simulate the dual-band microstrip antennas and assess its performance. Other software may also be employed to operate and assess the performance of the dual-band microstrip antennas. The dimensions of the dual-band microstrip antennas are determined by the resonant frequency and the dielectric constant value. In examples, the dual-band microstrip antennas are designed on an FR-4 substrate with a thickness of 1.6 mm and a dielectric constant of 4.3. The dual-band microstrip antenna dimensions and the positioning of the rectangular slots are chosen such that the antennas operate at the resonant frequencies of 2.5 GHz and 5.7 GHz.

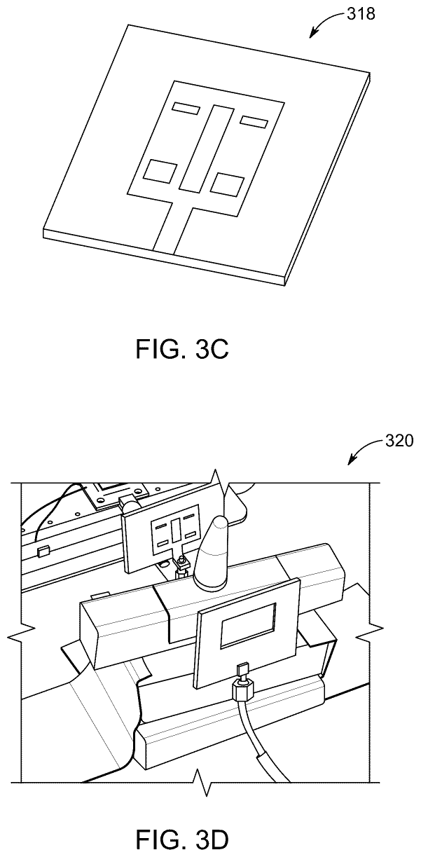

FIG. 4 shows a graphical representation 400 of simulated scattering parameters of two antennas when placed 7 cm apart and facing each other, as seen in FIG. 3D and when there are no samples between the two antennas, according to certain embodiments. As shown in FIG. 4, the behaviors of the reflections coefficients S₁₁ (plot line 402) and S₂₂ (plot line 404) show that both antennas are well-matched to the feeding microstrip line at 2.5 GHz and 5.7 GHz. The −10 dB impedance bandwidth ranges from 5.6 to 6.7 GHz at the higher operating band. Further, the plot line 406 represents reflection coefficients S₂₁.

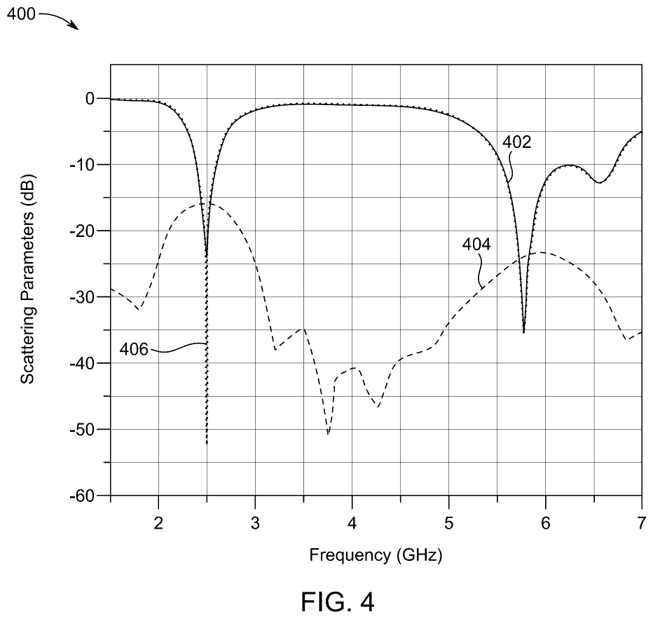

FIG. 5 shows a three-dimensional radiation pattern (directivity) 500 at 5.7 GHz when a first antenna 502 (which is an example of the first antenna 108) is excited while a second antenna 504 (which is an example of the second antenna 112) is terminated by a matched 50-ohm load, according to certain embodiments. In the example shown in FIG. 5, the far-field results show that the antenna radiation is directed from the first antenna 502 to the second antenna 504 with a maximum directivity of 4.51 dB. The directed radiated field may detect glucose in a solution which is placed between the first antenna 502 and the second antenna 504.

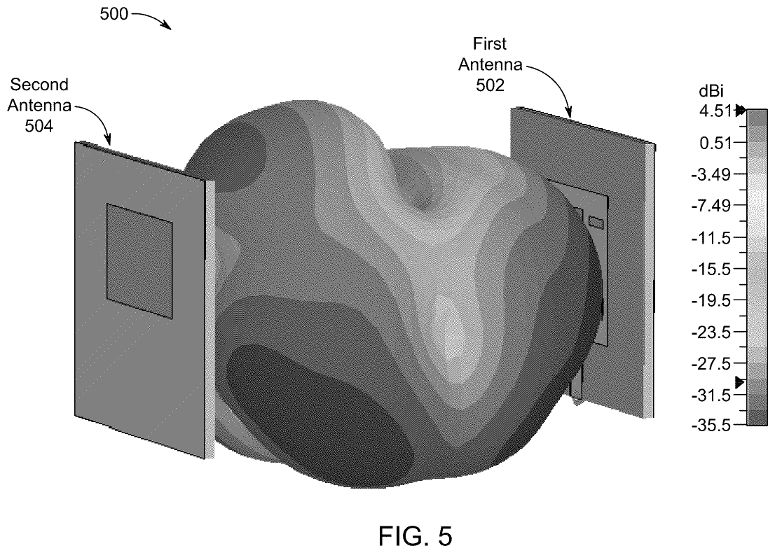

FIG. 6 shows peak electric field (near-field) distribution 600 at 5.7 GHz when a first antenna 602 (which is an example of the first antenna 108) is excited while a second antenna 604 (which is an example of the second antenna 112) is terminated by a matched 50-ohm load, according to certain embodiments. In the example shown in FIG. 6, adequate electric field intensity is observed between the first antenna 602 and the second antenna 604 over a virtual plane.

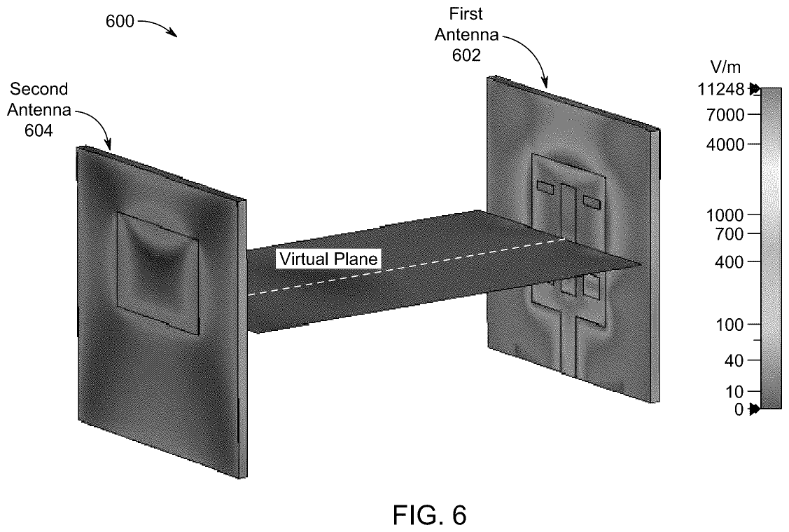

FIG. 7 shows a graphical representation 700 of an electric field (V/m) calculated along a virtual dash-line between the first antenna 602 and the second antenna 604, starting from the first antenna 602 to the second antenna 604, according to certain embodiments. The electric field is represented by plot line 702 in FIG. 7. In the example shown in FIG. 7, the electric field decreases as the distance between the first antenna 602 and the second antenna 604 is increased; with an adequate field value of about 85 V/m midway between the first antenna 602 and the second antenna 604 (i.e., at distance 35 mm from the first antenna 602) where a sample under test is to be placed.

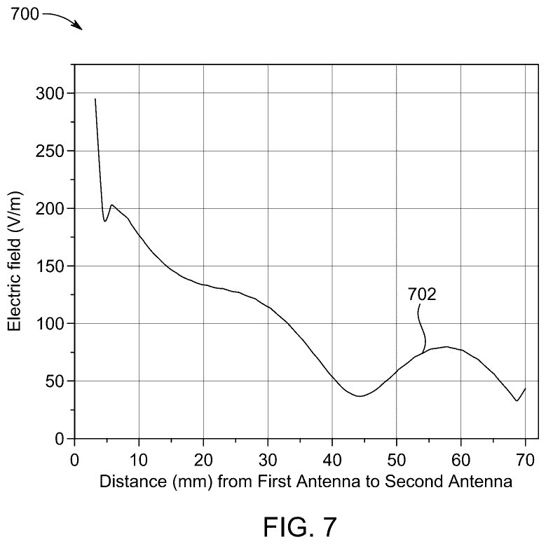

FIG. 8 shows a schematic diagram of an analog readout circuit 800, according to certain embodiments. In an example, the analog readout circuit 800 may be an example of the analog readout circuit 114. As shown in FIG. 8, the analog readout circuit 800 includes a low noise amplifier 802, a band-pass filter 804, and a radio frequency detector 806. The low noise amplifier 802 may provide good noise performance in addition to adequate amplification for the analog signal because the received RF (“radio frequency”) signal by the second antenna 604 is typically noisy, weak, and low in voltage amplitude (i.e., in the range of few hundreds μV to few mV). The present disclosure aims to provide a functioning glucose monitoring system holistically and to examine the practicality of the RF sensing mechanism.

For better signal conditioning, the filtering stage is implemented using the passive band-pass filter 804 which blocks undesirable low and high interference noise. The last stage of the analog readout circuit 800 is the radio frequency detector 806. The radio frequency detector 806 may convert the RF Alternative Current (AC) signal power into a Direct Current (DC) voltage level by envelope detection.

The band-pass filter may comprise any device which may pass signals or frequencies of a particular value range and block all signals or frequencies in excess of or below the filtering range. The band-pass filter may accomplish this through physical means, digital means, or any suitable combination thereof.

For the noise analysis of the analog readout circuit 800, the contributions of the low noise amplifier 802 and the band-pass filter 804 may be considered. The radio frequency detector 806 rectifies the RF signal, such that its contribution to the noise can be ignored. Therefore, the noise factor of the receiver side of the system 100 may be determined using Equation (1) provided below.

$${F}_{R} = {F}_{L N A} + \frac{{F}_{B P F} - 1}{{A}_{L N A}} \qquad ( 1 )$$

where F_{R} represents the noise factor of the receiver side, F_{LNA} represents the low noise amplifier noise figure, F_{BPF} represents the band-pass filter noise figure, and A_{LNA} represents the low noise amplifier voltage gain. In addition to the suppression of the noise by the band-pass filter 804, the noise figure of the band-pass filter 804 may be downscaled by the gain of the low noise amplifier 802 as described in Equation (1). Hence, the noise of the analog readout circuit 800 is mainly determined by the noise of the low noise amplifier 802.

The low noise amplifier may comprise any device which may amplify a low-power signal without significantly degrading its signal-to-noise ratio. The low noise amplifier may accomplish this through physical means, digital means, or any suitable combination thereof.

Examples of experiment setups, experiment methodologies, and experiment results are described below.

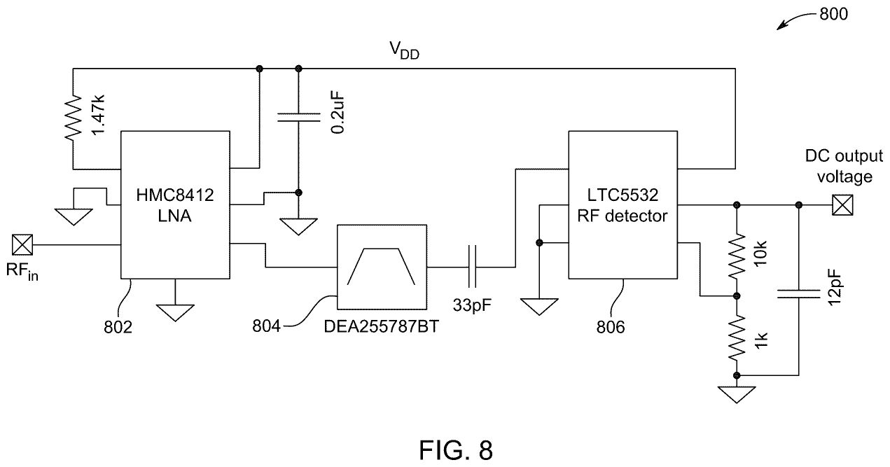

FIG. 9 describes an experimental setup 900 of the system 100 for monitoring the glucose (i.e., the microwave-based non-invasive glucose monitoring system), according to certain embodiments.

As shown in FIG. 9, an analog readout circuit 908 (which is an example of the analog readout circuit 114) is implemented on FR-4 printed circuit board (PCB) within an area of 4.32 cm by 1.78 cm, which is compatible with an Arduino nano board to be used for further signal processing. The analog readout circuit 908 includes GaAs-based LNA (HMC8412) from analog devices, which operates efficiently at 0.4-11 GHz and comes in Lead Frame Chip Scale Package (LFCSP), which is useful for system miniaturization. The low noise amplifier has a noise figure of 1.4 dB and an insertion loss of almost −0.3 dB, i.e., 94% efficient in delivering the input power.

A multilayer band-pass filter from TDK (DEA255787BT-2044A1) with a frequency range of 5.7 GHz to 6.0 GHz is used. The band-pass filter has about-1 dB insertion loss at 5.7 GHz. In addition, the radio frequency detector is implemented using a high precision wideband, 0.3-7 GHz, RF power detector (LTC5532) from Linear Technology. A radio frequency generator 906 (N5183A MXG) (which is an example of radio frequency generator 106) is used to generate the signal to be transmitted through a transmitting antenna. The radio frequency generator 906 can generate 100 kHz to 20 GHz signals with power range of −20 dBm up to 15 dBm. The analog readout circuit 908 consumes 306 mW at 5.1 V, which is supplied from an external power supply 902, as shown in the experimental setup 900 of FIG. 9.

The experimental setup 900 is to place a glucose-water solution sample between two antennas which are situated 7 cm apart from one another (depicted by reference numeral 910 in FIG. 9). A high-frequency signal of 5.7 GHz is generated and transmitted using a first antenna through the sample. On the other side, a second antenna receives the signal with different power levels compared to the transmitted signals and feeds the received signals to the analog readout circuit 908. The received signal is amplified, filtered, and then rectified. The DC voltage at the output terminals of the analog readout circuit 908 (i.e., output terminals of the RF power detector) is measured using a digital multimeter 904. The sample used for the experiment setup 900 is a plastic cup filled with deionized water and different glucose concentrations measured in mg/dL. The glucose weight is measured using a sensitive balance (Mettler Teldo AL204). For the experiment, 25 testing glucose-water samples were used to cover the range from 0-5 g/dL with steps of 200 mg/dL of glucose solution. The objective of the experiment is to study the relationship between the DC output voltage, the glucose concentration, and the input power levels of the system 100. The first step is to measure the output voltage for the glucose-free sample (i.e., pure water only) for various power levels. The input power is varied between 0 dBm to 15 dBm. Then, all other glucose samples are tested using 15 dBm input power to show the trend in the output voltage compared to the glucose concentration. In examples, more glucose is added for each new test sample, and the output voltage is measured. Due to the sensitivity of the system 100, three different voltage measurements of each sample are taken and then averaged out.

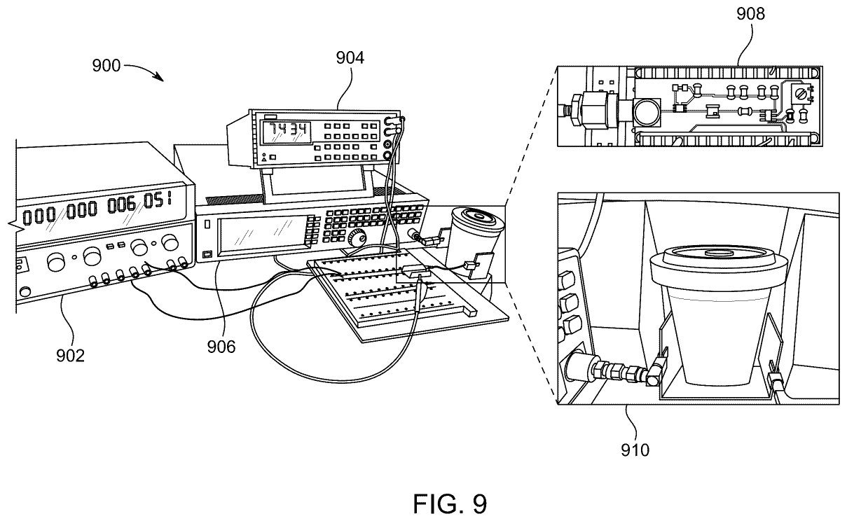

FIG. 10 shows a graphical representation 1000 of the DC output voltage trend versus glucose concentration, according to certain embodiments. In particular, FIG. 10 shows the trend of the DC output voltage level compared to glucose concentration for Pᵢₙ=15 dBm. The experimental data (represented by reference numeral “1002” in FIG. 10) represents the average dataset. The fitting line (represented by plot line “1004” in FIG. 10) is generated using the MATLAB curve fitting tool.

In examples, the experimental data set comprises the average values of the measured DC output voltages for each sample. The DC output voltage decreases with the increase in the glucose concentration. The MATLAB curve fitting tool is used to show the correlation between the DC output voltage and the glucose concentration. In an example, with second degree polynomial fitting and 95% confidence bounds, the DC output voltage may be mathematically expressed using Equation (2) provided below.

$${V}_{o u t} = 0.18 {G}^{2} - 2.11 G + 740.6 \qquad ( 2 )$$

where Vₒᵤₜ is represented in mV and G represents the glucose concentration level measured in g/dL. In addition to the effect of the glucose concentration, the experiment shows the effect of the input power on the sensitivity of the measured DC output voltage for different glucose-water samples.

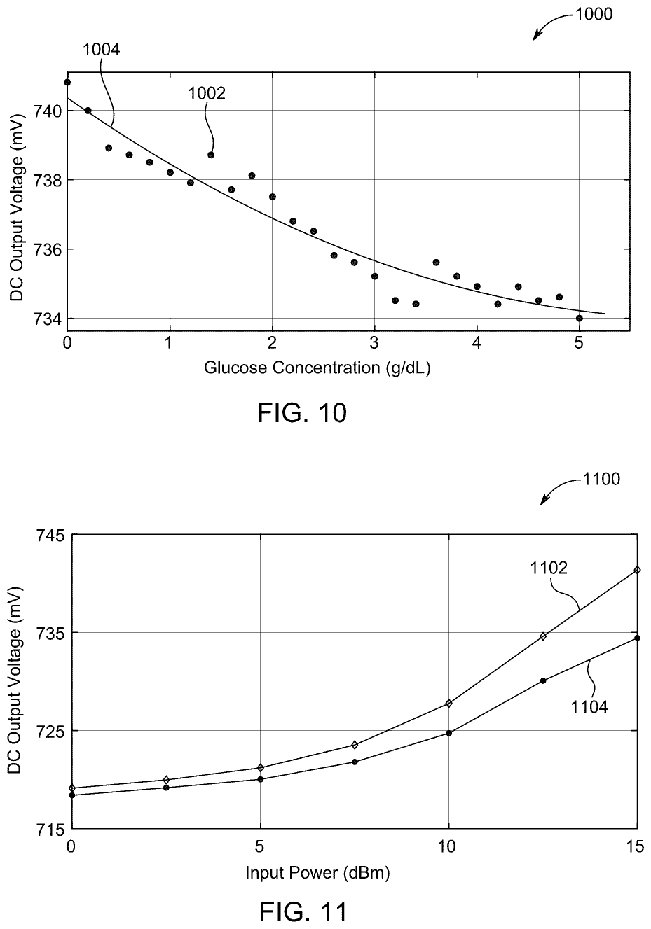

FIG. 11 shows a graphical representation 1100 illustrating a response of the DC output voltage to different power levels for glucose-free samples and 5 g/dL glucose concentration samples, according to certain embodiments.

In examples, as the input power increases, the measured DC output voltage increases, thus allowing the detection of other glucose concentration levels. For low input power levels, the difference between glucose-free samples (represented by plot line 1102 in FIG. 11) and 5 g/dL glucose concentration samples (represented by plot line 1104 in FIG. 11) is very small, which makes it difficult for an analog readout circuit to distinguish between close glucose concentration levels. For example, the voltage range between glucose-free samples and 5 g/dL glucose concentration samples is only 0.6 mV for Pᵢₙ=0 dBm. Consequently, the voltage sensitivity of the system 100 degrades as the input power decreases. Assuming that the system 100 detects five glucose concentration levels, from 0-5 g/dL with 1 g/dL step. For Pᵢₙ=0 dBm, the system 100 has only 120 μV of voltage room for each glucose concentration level. Therefore, at least 16 bit ADC stage is required when a 5V voltage supply microcontroller is used. As a result, the complexity and cost of the system 100 is substantially increased. Therefore, the voltage range (i.e., difference in DC output voltage between two samples (i.e., glucose-free and 5 g/dL glucose concentration samples) is a useful measurement to optimize the performance and system complexity and to identify the requirements of the system.

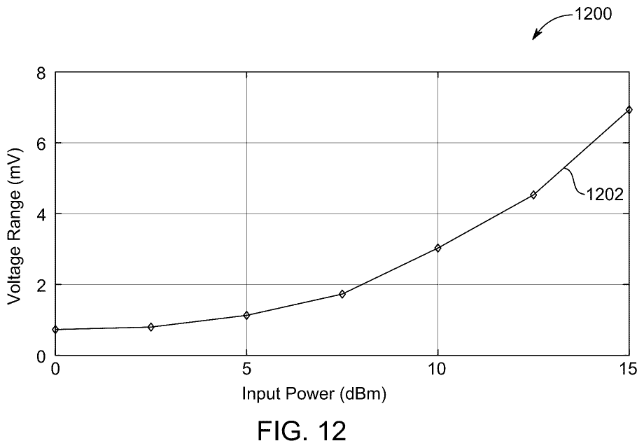

FIG. 12 shows a graphical representation 1200 of a voltage range between two samples including glucose-free samples and 5 g/dL glucose concentration samples versus different input power levels, according to certain embodiments. The voltage range is represented by plot line 1202 in FIG. 12.

The voltage sensitivity of the system 100 is interpreted as the amount of variation of the output variable (DC output voltage) with the variation of the input variable (glucose concentration) and may be quantified using Equation (3) provided below.

$$Sensitivity = \frac{1}{n} {\sum }_{i = 2}^{n} \vert \frac{{V}_{D {C}_{i}} - {V}_{D {C}_{i - 1}}}{{G}_{i} - {G}_{i - 1}} \vert \qquad ( 3 )$$

where n is the number of testing samples.

The experimental results show that there is a correlation between the glucose level and the DC output voltage. The system sensitivity is also studied for different input power levels. The system 100 achieved a voltage sensitivity of 2.65 μV/mgdL−1 using 15 dBm input power at 5.7 GHz frequency operation. The voltage sensitivity can be increased by providing more input power within an acceptable limit.

Table 1 provided below shows a comparison between the system 100 and conventional studies.

TABLE 1
Comparison of performance of the system 100 and conventional studies

| | | | | Glucose | | |
|---|---|---|---|---|---|---|
| | Biosensor | Operating | | Concentration | Sensitivity | System |
| Reference | Technology | Frequency | | (mg/dL) | (ΔX per 1 mg/dL) | Complexity |
| 2021, | Sub Terahertz | 110-170 | GHz | 70-145 | ΔS₂₁ = 0.13 dB | High |
| [Study A] | Waveguide | | | | | |
| 2013, | Rectangular | 1.91 | GHz | 0-2500 | ΔS₂₁ = 1.8e⁻⁵ dB | Medium |
| [Study B] | Cavity | | | (Sucrose | Δfᵣ = 0.015 kHz | |
| | | | | Solution) | | |
| 2019, | Millimeter- | 60-80 | GHz | 0-300 | ΔS₂₁ = 2.3 m dB | High |
| [Study C] | wave Horn | | | | Δ∠S₂₁ = 0.0153° | |
| | Antenna | | | | | |
| 2020, | Split Ring | 2-3 | GHz | 0-400 | ΔS₂₁ = 8e⁻⁵ dB | Medium |
| [Study D] | Oscillator | | | | | |
| 2015, | Complementary | 1.7 | GHz | 1000-9000 | Δfᵣ = 21.1 kHz | Medium |
| [Study E] | Port Resonator | | | (This value is | | |
| | | | | calculated. For | | |
| | | | | example, 10 | | |
| | | | | mg/mL is | | |
| | | | | equivalent to | | |
| | | | | 1000 mg/dL | | |
| | | | | and 10% of | | |
| | | | | glucose | | |
| | | | | concentration | | |
| | | | | is interpreted | | |
| | | | | as 10 mg/dL) | | |
| 2017, | Single Port | 4.8 | GHz | 0-1000 | Δfᵣ = 14 kHz | Medium |
| [Study F] | Resonator | | | | | |
| 2018, | Ultra-Wide | 1-18 | GHz | 20-70 | Δfᵣ = 446 kHz | High |
| [Study G] | Band Antenna | | | (This value is | | |
| | | | | calculated and | | |
| | | | | considering a | | |
| | | | | maximum | | |
| | | | | change of 70% | | |
| | | | | glucose | | |
| | | | | concentration) | | |
| System | Patch Antennas | 5.7 | GHz | 0-5000 | ΔV_{DC} = 2.65 μV | Low |

100

Study A-P. Kaurav, S. K. Koul, and A. Basu, “Non-invasive glucose measurement using sub-terahertz sensor, time domain processing, and neural network,” IEEE Sensors Journal, vol. 21, no. 18, pp. 20 002-20 009, 2021.
Study B-G. Gennarelli, S. Romeo, M. R. Scarf 1, and F. Soldovieri, “A microwave resonant sensor for concentration measurements of liquid solutions,” IEEE Sensors Journal, vol. 13, no. 5, pp. 1857-1864, 2013.
Study C-S. Hu, S. Nagae, and A. Hirose, “Millimeter-wave adaptive glucose concentration estimation with complex-valued neural networks,” IEEE Transactions on Biomedical Engineering, vol. 66, no. 7, pp. 2065-2071, 2019.
Study D-C. Jang, J.-K. Park, H.-J. Lee, G.-H. Yun, and J.-G. Yook, “Non-invasive fluidic glucose detection based on dual microwave complementary split ring resonators with a switching circuit for environmental effect elimination,” IEEE Sensors Journal, vol. 20, no. 15, pp. 8520-8527, 2020.
Study E-A. Ebrahimi, W. Withayachumnankul, S. F. Al-Sarawi, and D. Abbott, “Microwave microfluidic sensor for determination of glucose concentration in water,” in 2015 IEEE 15th Mediterranean Microwave Symposium (MMS), 2015, pp. 1-3.
Study F-V. Turgul and I. Kale, “Simulating the effects of skin thickness and fingerprints to highlight problems with non-invasive RF blood glucose sensing from fingertips,” IEEE sensors journal, vol. 17, no. 22, pp. 7553-7560, 2017.
Study G-A. K. Jha, Z. Akhter, N. Tiwari, K. M. Shafi, H. Samant, M. J. Akhtar, and M. Cifra, “Broadband wireless sensing system for non-invasive testing of biological samples,” IEEE Journal on Emerging and Selected Topics in Circuits and Systems, vol. 8, no. 2, pp. 251-259, 2018.

The experiments reported in study A, study C, study D, study E, and Study F were carried out using glucose-water samples and sucrose-water for study B, with an acceptable glucose concentration range. Meanwhile, study E used a high glucose concentration range of 1000-9000 mg/dL which can be useful for food applications but not for human blood glucose, which is typically between 50-500 mg/dL. Conversely, a small glucose concentration range is used in study A and study G. For the present disclosure, 0-5000 mg/dL of glucose concentration range is used which can be useful for medical and food applications. Although all references employ microwave-based techniques to detect the glucose, the measured parameter is different. For example, the glucose level is detected using the magnitude of the transmission coefficient (S21) in study A, study B, study C, and study D, the phase of S21 in study C, and/or the change in resonance frequency (fᵣ) in study E, study F, and study G. Further, the studies A-G reported in Table 1 are based on using a Vector Network Analyzer which is a very expensive and bulky equipment to measure S21 or the resonant frequency. This significantly hinders the objective of providing a portable and low-cost glucose sensing system. On the contrary, the system 100 noninvasively detects glucose levels based on the voltage level of the received signal, which is useful for achieving a portable glucose monitoring system. The system 100 achieves a voltage sensitivity of 2.65 μV/mgdL−1 based on the Equation (2).

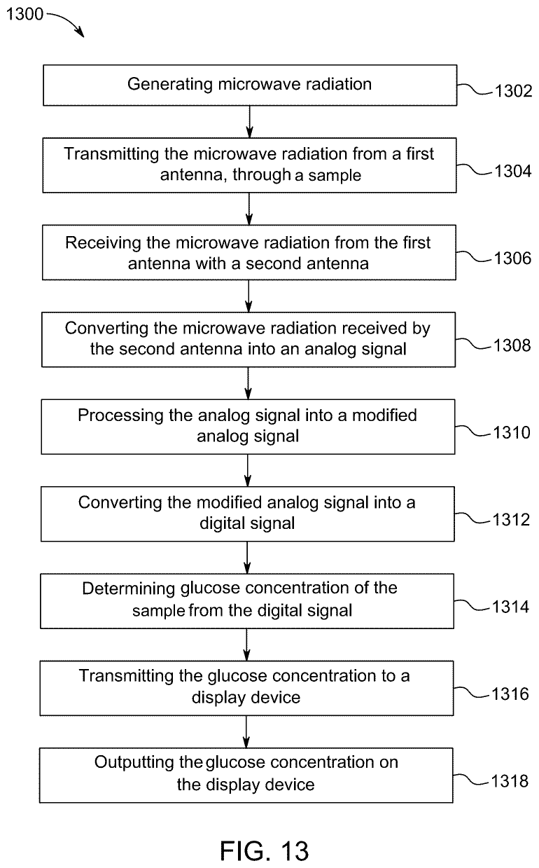

FIG. 13 illustrates a flowchart 1300 for monitoring glucose concentration, according to aspects of the present disclosure.

At step 1302 of the flowchart 1300, microwave radiation may be generated. In an implementation, the radio frequency generator 106 may be configured to generate microwave radiation.

At step 1304 of the flowchart 1300, the microwave radiation may be transmitted from a first antenna, through a sample. In an implementation, the radio frequency transmitter 104 may transmit the microwave radiation from the first antenna 108, through the sample.

At step 1306 of the flowchart 1300, the microwave radiation may be received from the first antenna with a second antenna. In an implementation, the second antenna 112 of the radio frequency receiver 110 may receive the microwave radiation from the first antenna 108. The first antenna 108 and the second antenna 112 are microstrip antennas. The microstrip antennas may comprise a ground plate, a dielectric substrate disposed on the ground plate, and a conductive pattern disposed on the dielectric substrate. In examples, the conductive pattern includes a rectangular patch with a plurality of slots disposed on the patch. The plurality of slots may include two horizontally oriented slots disposed on a first side of the antenna, two horizontally oriented slots disposed on a second side of the antenna, and one vertically oriented slot disposed on the antenna between the first and second sides of the antenna. The plurality of slots may be disposed on one or both sides of the microstrip antennas.

At step 1308 of the flowchart 1300, the microwave radiation received by the second antenna may be converted into an analog signal. In an implementation, the radio frequency receiver 110 is configured to convert the microwave radiation passed through the sample and received by the second antenna 112 into the analog signal.

At step 1310 of the flowchart 1300, the analog signal may be processed into a modified analog signal. In an implementation, the analog readout circuit 114 is configured to process the analog signal into the modified analog signal.

At step 1312 of the flowchart 1300, the modified analog signal may be converted into a digital signal. In an implementation, the digital signal processing circuit 116 is configured to convert the modified analog signal into the digital signal.

At step 1314 of the flowchart 1300, the glucose concentration of the sample may be determined from the digital signal. In an implementation, the digital signal processing circuit 116 is configured to determine the glucose concentration of the sample from the digital signal.

At step 1316 of the flowchart 1300, the glucose concentration may be transmitted to a display device. In an implementation, the digital signal processing circuit 116 is configured to transmit the glucose concentration to the display device 212.

At step 1318 of the flowchart 1300, the glucose concentration may be output on the display device. In an implementation, the display device 212 is configured to visually output the glucose concentration.

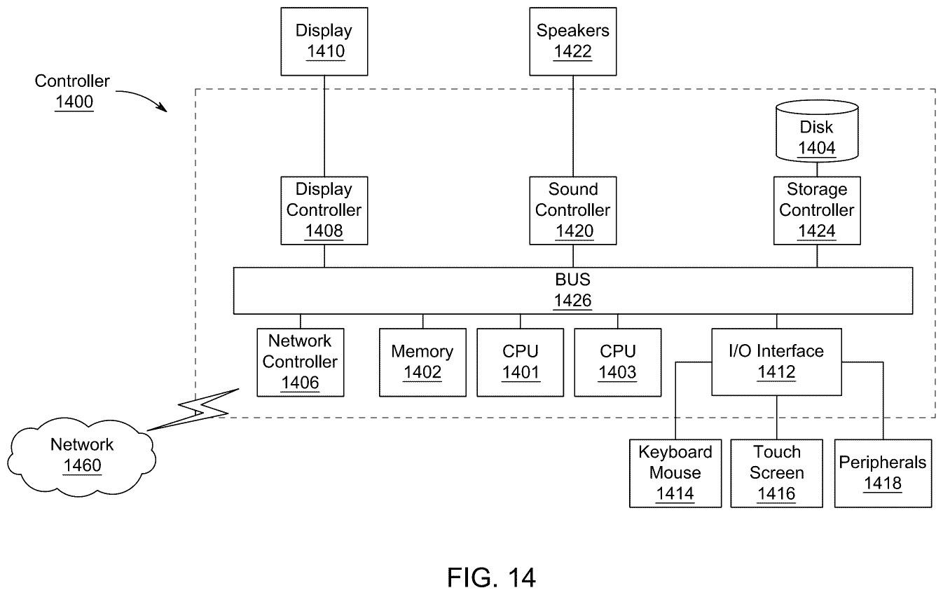

FIG. 14 is an illustration of a non-limiting example of details of computing hardware used in the computing system.

Next, further details of the hardware description of the computing environment according to exemplary embodiments is described with reference to FIG. 14. FIG. 14 is an illustration of a non-limiting example of details of computing hardware used in the computing system, according to exemplary aspects of the present disclosure. In FIG. 14, a controller 1400 is described which is a computing device and includes a CPU 1401 which performs the processes described above/below. The process data and instructions may be stored in memory 1402. These processes and instructions may also be stored on a storage medium disk 1404 such as a hard drive (HDD) or portable storage medium or may be stored remotely.

Further, the claims are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computing device communicates, such as a server or computer.

Further, the claims may be provided as a utility application, background daemon, or component of an operating system, or combination thereof, executing in conjunction with CPU 1401, 1403 and an operating system such as Microsoft Windows 7, Microsoft Windows 10, Microsoft Windows 11, UNIX, Solaris, LINUX, Apple MAC-OS and other systems known to those skilled in the art.

The hardware elements used in order to achieve the computing device may be realized by various circuitry elements, known to those skilled in the art. For example, CPU 1401 or CPU 1403 may be a Xenon or Core processor from Intel of America or an Opteron processor from AMD of America, or may be other processor types that would be recognized by one of ordinary skill in the art. Alternatively, the CPU 1401, 1403 may be implemented on an FPGA, ASIC, PLD or using discrete logic circuits, as one of ordinary skill in the art would recognize. Further, CPU 1401, 1403 may be implemented as multiple processors cooperatively working in parallel to perform the instructions of the inventive processes described above.

The computing device in FIG. 14 also includes a network controller 1406, such as an Intel Ethernet PRO network interface card from Intel Corporation of America, for interfacing with network 1460. As can be appreciated, the network 1460 can be a public network, such as the Internet, or a private network such as an LAN or WAN network, or any combination thereof and can also include PSTN or ISDN sub-networks. The network 1460 can also be wired, such as an Ethernet network, or can be wireless such as a cellular network including EDGE, 3G, 4G and 5G wireless cellular systems. The wireless network can also be WiFi, Bluetooth, or any other wireless form of communication that is known.

The computing may device further include a display controller 1408, such as a NVIDIA Geforce GTX or Quadro graphics adaptor from NVIDIA Corporation of America for interfacing with display 1410, such as a Hewlett Packard HPL2445w LCD monitor. A general purpose I/O interface 1412 interfaces with a keyboard and/or mouse 1414 as well as a touch screen panel 1416 on or separate from display 1410. General purpose I/O interface also connects to a variety of peripherals 1418 including printers and scanners, such as an OfficeJet or DeskJet from Hewlett Packard.

A sound controller 1420 is also provided in the computing device such as Sound Blaster X-Fi Titanium from Creative, to interface with speakers/microphone 1422 thereby providing sounds and/or music. The general purpose storage controller 1424 connects the storage medium disk 1404 with communication bus 1426, which may be an ISA, EISA, VESA, PCI, or similar, for interconnecting all of the components of the computing device. A description of the general features and functionality of the display 1410, keyboard and/or mouse 1414, as well as the display controller 1408, storage controller 1424, network controller 1406, sound controller 1420, and general purpose I/O interface 1412 is omitted herein for brevity as these features are known.

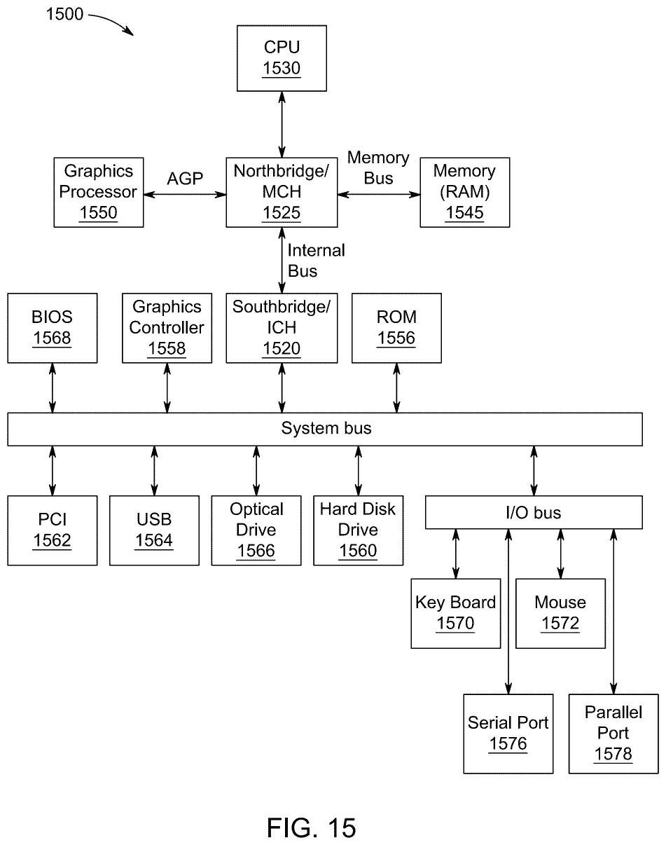

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown on FIG. 15.

FIG. 15 shows a schematic diagram of a data processing system 1500 for performing the functions of the exemplary embodiments. The data processing system 1500 is an example of a computer in which code or instructions implementing the processes of the illustrative embodiments may be located.

In FIG. 15, data processing system 1500 employs a hub architecture including a north bridge and memory controller hub (NB/MCH) 1525 and a south bridge and input/output (I/O) controller hub (SB/ICH) 1520. The central processing unit (CPU) 1530 is connected to NB/MCH 1525. The NB/MCH 1525 also connects to the memory 1545 via a memory bus and connects to the graphics processor 1550 via an accelerated graphics port (AGP). The NB/MCH 1525 also connects to the SB/ICH 1520 via an internal bus (e.g., a unified media interface or a direct media interface). The CPU Processing unit 1530 may contain one or more processors and even may be implemented using one or more heterogeneous processor systems.

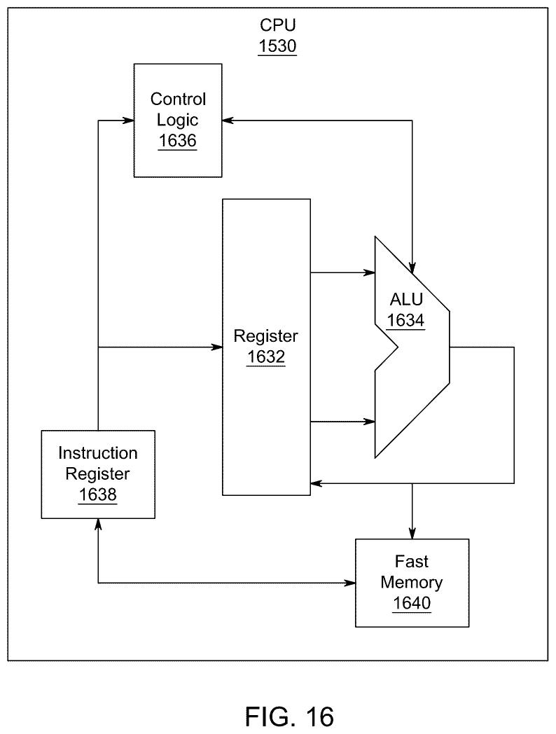

For example, FIG. 16 shows one implementation of CPU 1530. In one implementation, the instruction register 1638 retrieves instructions from the fast memory 1640. At least part of these instructions are fetched from the instruction register 1638 by the control logic 1636 and interpreted according to the instruction set architecture of the CPU 1530. Part of the instructions can also be directed to the register 1632. In one implementation, the instructions are decoded according to a hardwired method, and in another implementation, the instructions are decoded according to a microprogram that translates instructions into sets of CPU configuration signals that are applied sequentially over multiple clock pulses. After fetching and decoding the instructions, the instructions are executed using the arithmetic logic unit (ALU) 1634 that loads values from the register 1632 and performs logical and mathematical operations on the loaded values according to the instructions. The results from these operations can be fed back into the register and/or stored in the fast memory 1640. According to certain implementations, the instruction set architecture of the CPU 1530 can use a reduced instruction set architecture, a complex instruction set architecture, a vector processor architecture, and/or a very large instruction word architecture. Furthermore, the CPU 1530 can be based on the Von Neuman model or the Harvard model. The CPU 1530 can be a digital signal processor, an FPGA, an ASIC, a PLA, a PLD, or a CPLD. Further, the CPU 1530 can be an ×86 processor by Intel or by AMD; an ARM processor, a Power architecture processor by, e.g., IBM; a SPARC architecture processor by Sun Microsystems or by Oracle; or other known CPU architecture.

Referring again to FIG. 16, the data processing system 1500 can include that the SB/ICH 1520 is coupled through a system bus to an I/O Bus, a read only memory (ROM) 1556, universal serial bus (USB) port 1564, a flash binary input/output system (BIOS) 1568, and a graphics controller 1558. PCI/PCIe devices can also be coupled to SB/ICH 1520 through a PCI bus 1562.

The PCI devices may include, for example, Ethernet adapters, add-in cards, and PC cards for notebook computers. The Hard disk drive 1560 and CD-ROM 1556 can use, for example, an integrated drive electronics (IDE) or serial advanced technology attachment (SATA) interface. In one implementation, the I/O bus can include a super I/O (SIO) device.

Further, the hard disk drive (HDD) 1560 and optical drive 1566 can also be coupled to the SB/ICH 1520 through a system bus. In one implementation, a keyboard 1570, a mouse 1572, a parallel port 1578, and a serial port 1576 can be connected to the system bus through the I/O bus. Other peripherals and devices that can be connected to the SB/ICH 1520 using a mass storage controller such as SATA or PATA, an Ethernet port, an ISA bus, a LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes in battery sizing and chemistry or based on the requirements of the intended back-up load to be powered.

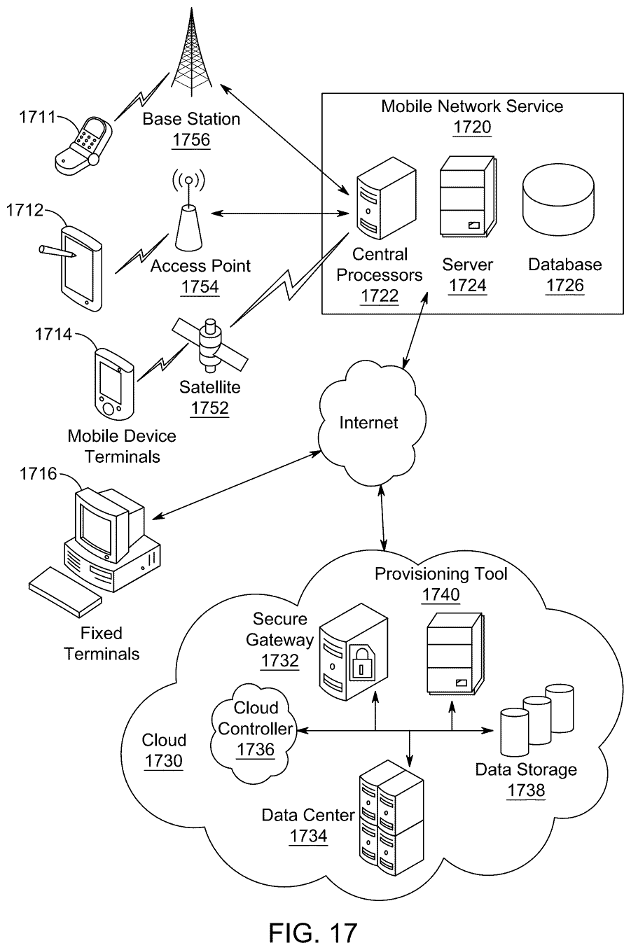

The functions and features described herein may also be executed by various distributed components of a system. For example, one or more processors may execute these system functions, wherein the processors are distributed across multiple components communicating in a network. The distributed components may include one or more client and server machines, which may share processing, as shown by FIG. 17, in addition to various human interface and communication devices (e.g., display monitors, smart phones, tablets, personal digital assistants (PDAs)).

More specifically, FIG. 17 illustrates client devices including a smart phone 1711, a tablet 1712, a mobile device terminal 1714 and fixed terminals 1716. These client devices may be commutatively coupled with a mobile network service 1720 via base station 1756, access point 1754, satellite 1752 or via an internet connection. Mobile network service 1720 may comprise central processors 1722, a server 1724 and a database 1726. Fixed terminals 1716 and mobile network service 1720 may be commutatively coupled via an internet connection to functions in cloud 1730 that may comprise security gateway 1732, data center 1734, cloud controller 1736, data storage 1738 and provisioning tool 1740. The network may be a private network, such as a LAN or WAN, or may be a public network, such as the Internet. Input to the system may be received via direct user input and received remotely either in real-time or as a batch process. Additionally, some implementations may be performed on modules or hardware not identical to those described. Accordingly, other implementations are within the scope that may be claimed.

The above-described hardware description is a non-limiting example of corresponding structure for performing the functionality described herein.

Numerous modifications and variations of the present disclosure are possible in light of the above teachings. It is therefore to be understood that within the scope of the appended claims, the invention may be practiced otherwise than as specifically described herein.

1. A system for monitoring glucose concentration, the system comprising:
a sample holder configured to contain a sample;
a radio frequency transmitter configured to emit microwave radiation through the sample, the radio frequency transmitter comprising a radio frequency generator and a first antenna;
a radio frequency receiver configured to receive microwave radiation transmitted from the radio frequency transmitter and passed through the sample, and convert the microwave radiation passed through the sample to an analog signal, the radio frequency receiver comprising a second antenna;
wherein, the first and second antennas are microstrip antennas having a sample side and an outward side, the microstrip antennas further comprising:
a ground plate;
a dielectric substrate disposed on the ground plate; and
a conductive pattern disposed on the dielectric substrate, wherein:
the conductive pattern on the sample side of the microstrip antenna comprises a rectangular patch with a plurality of slots disposed on the patch, the plurality of slots being configured to expose regions of the dielectric substrate; and
the rectangular patch is symmetrical about a long axis, the long axis defining first and second sides of the sample side of the rectangular patch, wherein the sample side of the rectangular patch comprises a plurality of slots including:
two horizontal slots symmetrically oriented at a top half of the rectangular patch;
two horizontal slots symmetrically oriented at a bottom half of the rectangular patch; and
a vertically oriented slot disposed on the long axis;
an analog readout circuit configured to receive the analog signal and process the analog signal into a modified analog signal; and
a digital signal processing circuit configured to:
receive the modified analog signal;
convert the modified analog signal into a digital signal; and
determine the glucose concentration of the sample from the digital signal.

2. The system of claim 1, wherein the conductive pattern on the outward side of the microstrip antenna comprises a rectangular patch with a single slot disposed on the patch, the slot being configured to expose a region of the dielectric substrate.

3. The system of claim 2, wherein the rectangular patch is symmetrical about a long axis, the long axis defining first and second sides of the outward side of the rectangular patch, wherein the slot is positioned across the first side and the second side of the rectangular patch:

4. The system of claim 1, wherein:
the two horizontal slots symmetrically oriented at the top half of the rectangular patch are the same size as one another;
the two horizontal slots symmetrically oriented at a bottom half of the rectangular patch are the same size as one another; and
the vertically oriented slot disposed on the long axis is larger than any of the horizontal slots on the rectangular patch.

5. The system of claim 4, wherein the microstrip antennas are configured to operate at the resonant frequencies of 2.5 GHz and 5.7 Ghz.

6. The system of claim 1, wherein the analog readout circuit comprises:
a low noise amplifier;
a band-pass filter; and
a radio frequency detector.

7. The system of claim 6, wherein the low noise amplifier is configured to operate between 0.4 GHz and 11 GHz.

8. The system of claim 6, wherein the band-pass filter is configured to operate in the frequency range of 5.7 GHz to 6.0 GHz.

9. The system of claim 6, wherein the radio frequency detector comprises a high precision wideband radio frequency power detector.

10. The system of claim 6, wherein the high precision wideband radio frequency power detector is configured to detect radio frequencies in the range of 0.3 GHz to 7 GHz.

11. The system of claim 1, wherein the digital signal processing circuit comprises:
an analog to digital signal converter;
a digital signal processing unit;
a wireless communication unit; and
a display device.

12. The system of claim 11, wherein the wireless communication unit comprises a Bluetooth transmitter.

13. The system of claim 12, wherein the display device comprises a Bluetooth receiver and a screen, the display device being configured to visually output the glucose concentration.

14. A system for monitoring glucose concentration, the system comprising:
a radio frequency generator;
a first antenna configured to transmit microwave radiation through a sample;
a second antenna configured to receive microwave radiation transmitted by the first antenna and convert the microwave radiation into an analog signal;
an analog readout circuit configured to receive the analog signal and process the analog signal into a modified analog signal; and
a digital signal processing circuit configured to:
receive the modified analog signal;
convert the modified analog signal into a digital signal; and
determine the glucose concentration of the sample from the digital signal.

15. The system of claim 14, wherein the first and second antennas are microstrip antennas, the microstrip antennas comprising:
a ground plate;
a dielectric substrate disposed on the ground plate; and
a conductive pattern disposed on the dielectric substrate.

16. The system of claim 15, wherein the conductive pattern comprises a rectangular patch with a plurality of slots disposed on the patch, the plurality of slots comprising:
two horizontally oriented slots disposed on a first side of the microstrip antenna;
two horizontally oriented slots disposed on a second side of the microstrip antenna; and
one vertically oriented slot disposed on the microstrip antenna between the first and second sides of the microstrip antenna, wherein the plurality of slots are configured to expose regions of the dielectric substrate such that the microstrip antennas operate at the resonant frequencies of 2.5 GHz and 5.7 Ghz.

17. The system of claim 16, wherein the analog readout circuit comprises:
a low noise amplifier;
a band-pass filter; and
a radio frequency detector.

18. A method for monitoring glucose concentration, the method comprising:
generating microwave radiation;
transmitting the microwave radiation from a first antenna, through a sample;
receiving the microwave radiation from the first antenna with a second antenna;
converting the microwave radiation received by the second antenna into an analog signal;
processing the analog signal into a modified analog signal;
converting the modified analog signal into a digital signal;
determining the glucose concentration of the sample from the digital signal;
transmitting the glucose concentration to a display device; and
outputting the glucose concentration on the display device.

19. The method of claim 18, wherein the first and second antennas are microstrip antennas, the microstrip antennas comprising:
a ground plate;
a dielectric substrate disposed on the ground plate; and
a conductive pattern disposed on the dielectric substrate.

20. The method of claim 19, wherein the conductive pattern comprises a rectangular patch with a plurality of slots disposed on the patch, the plurality of slots comprising:
two horizontally oriented slots disposed on a first side of the antenna;
two horizontally oriented slots disposed on a second side of the antenna; and
one vertically oriented slot disposed on the antenna between the first and second sides of the antenna.
